(19) United States
(12) Patent Application Publication
(10) Pub. No.: US 2004/0015116 A1
(43) Pub. Date: Jan. 22, 2004

(54) Firm-contact apparel prosthesis for tremor suppression and method of use thereof

(76) Inventor: Charles Adrian Handforth, Los Angeles, CA (US)

Correspondence Address: DYKEMA GOSSETT PLLC, FRANKLIN SQUARE, THIRD FLOOR WEST, 1300 I STREET, NW, WASHINGTON, DC, 20005, US

(21) Appl. No.: 10/199,708

(22) Filed: Jul. 22, 2002

(51) Int. Cl. A61F 13/00; A61F 13/06

(52) U.S. Cl. 602/61

(57) ABSTRACT

A firm-contact apparel prosthesis and method for tremor suppression for applying a consistent and constant pressure around the entire circumference of a part of the body. The prosthesis is made of a band of elastic material that is continuous and firm, or may be made of an article of clothing having one or more bands integrally formed in the article of clothing. Multiple prosthetics may be used at the same time. The method of tremor suppression requires the steps of applying pressure evenly and circumferentially about a part of the body, for extended periods of time or for intervals of time.

### BACKGROUND OF THE INVENTION

This invention relates to a prosthesis for tremor suppression and more particularly to a firm-contact apparel prosthesis that provides circumferential pressure on a part of the body so as to reduce tremors.

### DESCRIPTION OF THE RELATED ART

Tremors affect a large population of people every day. A simple method of classifying tremor disorders is to consider them as either resting tremor, postural tremors or kinetic tremors. The subject of this disclosure is for the treatment for alleviating or suppressing the effects of tremors, in particular those affecting the upper extremities of the body. These tremors not only make the completion of tasks difficult, but they also can be a source of embarrassment. A number of therapies exist for the treatment of tremors, but most have associated side effects or cost penalties which inhibit their use.

The firm-contact apparel prosthesis for tremor suppression of the present invention is an inexpensive, risk free, non-surgical, reversible means for suppressing tremors.

There are currently a number of different types of treatment of tremors such as pharmacological methods, surgical methods, nerve stimulation, cell implantations and orthotic devices.

Tremors, such as those associated with Parkinson's disease, are treated with one or a combination of drug therapies consisting of carbidopa, levidopa trihexyphenidyl, amantadur or tolcapone. Side effects of these drug treatments may include memory impairment, hallucinations, dry mouth, urinary difficulties or blurred vision.

Essential tremors are pharmaceutically treated with propanolol or primidone. Side effects of these drugs may include dizziness, fatigue, depression, ataxia, nausea, or sleepiness.

There are also surgical methods such as thalamotomy and pallidotomy that may be used in the treatment of tremors. Either of these methods consist of lesioning a portion of the brain, such as the thalamus or the globus pallidus. The lesioning is an irreversible action and these procedures carry the risk associated with neurosurgery such as inadvertent injury to nearby structure, hemorrhage, or infection.

Thalamic stimulation provides an alternative to lesioning, yet is reversible and adjustable. This type of brain stimulation is costly, and carries those risks associated with brain surgery such as infection, seizure and stroke.

Cell implantation is another known treatment wherein cells from a healthy source are transplanted to the site of unhealthy cells. The cell source for such a treatment can be fetal, porcine, or epithelial cells. Such an implantation also has risks associated with surgery.

Orthotic devices have also been developed in the treatment of tremors. A tremor-suppression orthosis is used to apply viscous resistance to motion of a body part in flexion and extension. The orthosis reduces tremor amplitude and is small enough to be worn under a person's clothing.

The firm-contact apparel prosthesis for tremor suppression is a device for exerting direct sustained pressure selectively on one or more parts of the body. The prosthesis is comprised of a sleeve, band or combination thereof that applies pressure around a body extremity such as an arm or wrist, but not limited thereto.

### BRIEF DESCRIPTION OF THE INVENTION

An object of the present invention is to provide a method and device for suppressing tremors in various parts of the body.

The objective is achieved by a firm-contact apparel prosthesis made of a band of elastic material that is continuous and firm which applies an even and constant pressure about the circumference of a person's appendage. Multiple prosthetics may be used simultaneously for a single period of time, or for various intervals of time.

### BRIEF DESCRIPTION OF THE DRAWINGS

FIG. 1 is a perspective view of a first embodiment of the firm-contact apparel prosthesis for tremor suppression of the present invention;

FIG. 2 is a perspective view of a second embodiment of the present invention showing the invention with a fastener;

FIG. 3 is a top plan view of a third embodiment of the present invention showing the invention in the form of a glove;

FIG. 4 is a perspective view of a fourth embodiment of the present invention showing the invention in the form of a sleeve; and

FIG. 5 is a cross-sectional view of the fourth embodiment taken along line A-A in FIG. 4.

### DETAILED DESCRIPTION OF THE INVENTION

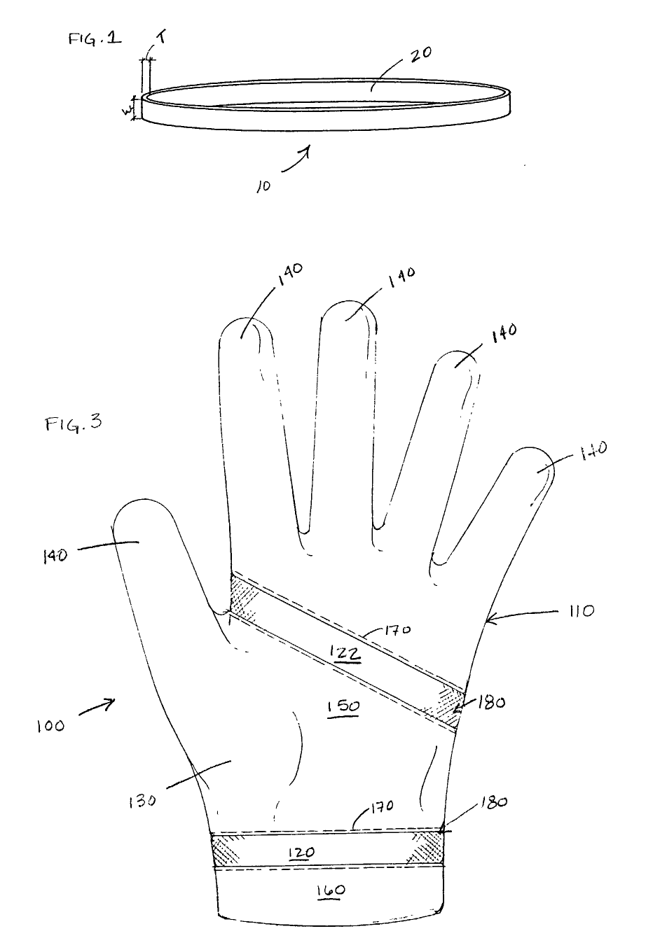

FIG. 1 illustrates a first embodiment of the firm-contact apparel prosthesis 10 which is comprised of a single, unitary band 20. The band 20 is made of a flexible material having elastic characteristics. While the preferred embodiment may be made of rubber, any type of composite material, plastic, cloth or mesh, other flexible materials with memory or having elastic qualities are foreseeable. The firm-contact apparel prosthesis 10 may also take on a variety of shapes and forms.

The band 20 can be of varying sizes to facilitate placement on different size appendages of a person suffering from tremors. The single, unitary band 20 has a circumference, and a thickness T as well as a width W. The thickness T and width W may be adjusted to provide a comfortable fit for the person wearing the band 20. For example, a larger person may require a greater thickness T, or a larger width W or circumference in order to achieve the same results as a smaller, more petite person. Also, the adjustable nature of the band 20 allows for the same band 20 to be used in various locations on the body such as about the elbow, the wrist or palm of a person's body.

The thickness T and width W may also be dictated by the type of material of the band 20. A stronger material may allow for a thinner thickness T of the band, while a weaker material may require a thicker thickness T so as to avoid breaking the band when it is in use.

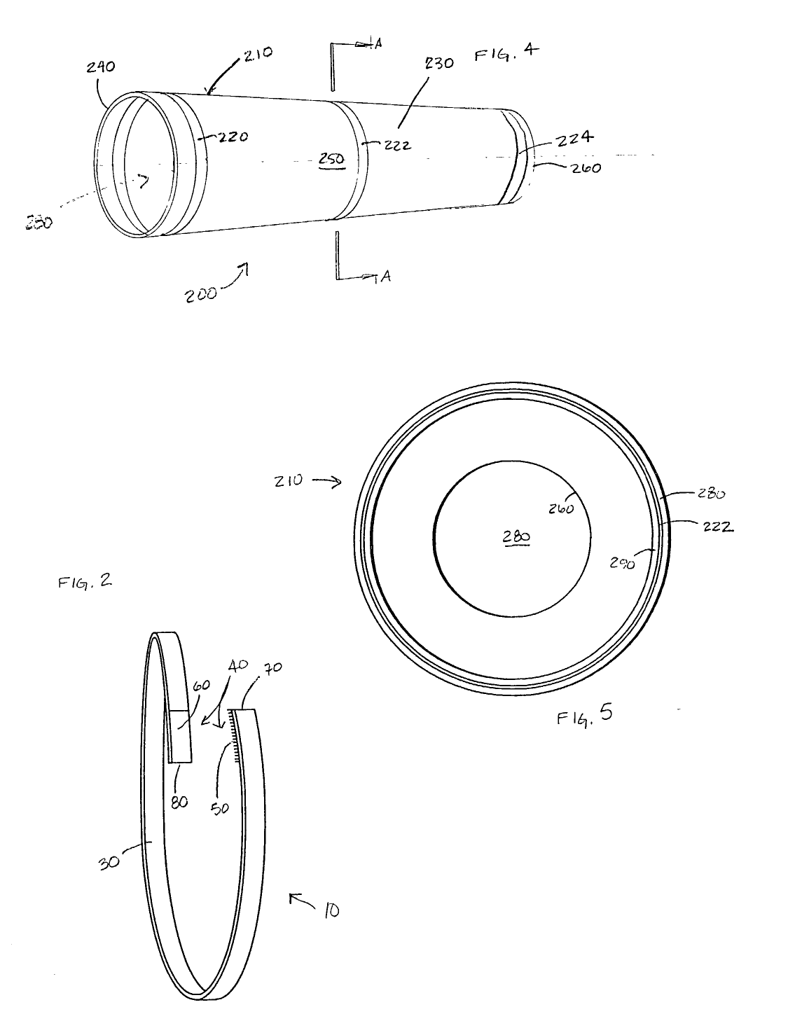

Each band 20 can be either one single continuous band, as shown in FIG. 1, or may be comprised of a strap 30 of material having a fastener 40. The fastener 40 has a first section 50 at a first end 70 of the strap 30, and a second section 60 at the second end 80 of the strap 30, as shown in FIG. 2.

In the second embodiment of the firm-contact apparel prosthesis 10, each section 50, 60 of the fastener 40 can be connected together so the strap 30 forms a band 20. An advantage of this second embodiment of the band 20 is that it is adjustable to different sizes thereby accommodating various types of people and various types of body parts.

The fastener 40 can be any common type fastener known in the art having two separate sections such as hook and loop, snap, hook and eye, clip, adhesive, button, and even zipper, but the list is not all inclusive, merely illustrative as other types of fasteners are foreseeable. The two ends 70, 80 of the strap 30 may not have a fastener 40 located thereon, and may be tied together to form the band 20 to the appropriate size and shape desired.

The band 20 is used to apply a constant, circumferential pressure about a desire body part so as to suppress undesired tremors of the body part about which the band 20 is applies, as well as other body parts in the vicinity of the band 20, although not directly adjacent thereto.

A third embodiment of the firm-contact apparel prosthesis 100 is illustrated in FIG. 3, wherein the prosthesis 100 is in the form of a glove 110. The glove 110 has a main body 130 with appendage sleeves or fingers 140. There may be any number of bands enmeshed with the glove 110, but two are shown: a first band 120, and a second band 122.

The first band 120 is located in the wrist portion 160 of the glove 110 and is enclosed within an inner pocket 180 formed within the glove 110 by any convention means such as stitches 170, glue or simply integrally formed therein depending upon what material the glove 110 is made of.

A second band 122 is also contained within another inner pocket 180 located at the palm portion 150 of the glove 110. The second band 122 may be located anywhere whithin the palm portion 150 of the glove 110, as the location will be dictated by the needs of each individual wearer or user of the glove 110.

FIG. 4 shows a fourth embodiment of the firm-contact apparel prosthesis 200 in the form of a sleeve 210. The sleeve 210 can be adapted to be worn on either the leg or the arm, but will be described as relevant to the arm.

Within the body 230 of the sleeve 210, are a plurality of bands 220, 222, 224, each strategically located along the length of the sleeve 210 and encircling the circumference thereof. The sleeve 210 has a central aperture 280 through which a user slips their arm.

The first band 220 is shown at the first end portion 240 of the sleeve 210 and would accommodate pressure being applied to the upper arm of a person wearing the sleeve 210.

The second band 222 is located within the middle portion 250 of the sleeve 210 thereby applying pressure to the elbow region of a person wearing the sleeve 210.

The third band 224 is formed within or connected to the sleeve 210 at the second end portion 260 of the sleeve 210 which is located within the region of the wrist of a person wearing the sleeve 210 on their arm.

While three bands 220, 222, 224 are shown in three general locations 240, 250, 260, it is conceivable that any desired number of bands can be made in the garment and any desired location long the body 230 thereof. The bands therein can be of the same or varying widths and thicknesses, and have different circumferences depending upon their location on the sleeve 210. A single band may also extend the entire length of the sleeve 210.

A cross-sectional view of the sleeve 210 is taken through the second band 222 to show the band itself 222 being held between an outer layer of sleeve material 280 and an inner layer 290. The two layers 280, 290 of prosthetic material maintain the band 222 within a specific location so pressure can be applied to a specific region of a user's body.

1. A prosthesis for tremor suppression comprising:
a band having a circumference;
wherein said band provides a constant pressure throughout the entire circumference thereof, and said pressure suppresses tremors.

2. A prosthesis for tremor suppression comprising:
said band is made of elastomeric material.

3. The prosthesis for tremor suppression according to claim 2, wherein:
said band is one unitary, continuous strap.

4. The prosthesis for tremor suppression according to claim 2, wherein:
said band is a strap with a fastener for facilitating placement of said band around an appendage.

5. The prosthesis for tremor suppression according to claim 4, wherein:
said band has a first end and a second end;
a first section of said fastener is attached at said first end of said band;
a second section of said fastener is attached at said second end of said band;
wherein said first section and said second section matingly join to form a connection between said two sections forming a continuous band when said first section of said band is attached with said second section of said band.

6. A firm-contact apparel prosthesis for tremor suppression comprising:
an article of clothing having at least one band with a circumference;
said at least one band is made of elastomeric material;
said at least one band is attached to said article of clothing;
wherein said at least one band provides a constant pressure throughout the entire circumference thereof.

7. The firm-contact apparel prosthesis for tremor suppression according to claim 6, wherein:
said article of clothing has an internal layer and an external layer;
said at least one band is between said internal layer and said external layer.

8. The firm-contact apparel prosthesis for tremor suppression according to claim 7, wherein:
said article of clothing is a glove; and
said at least on band is located in said palm area of said glove.

9. The firm-contact apparel prosthesis for tremor suppression according to claim 7, wherein:
said article of clothing is a sleeve.

10. The firm-contact apparel prosthesis for tremor suppression according to claim 8, wherein:
said band is located in a central portion of said sleeve.

11. The firm-contact apparel prosthesis for tremor suppression according to claim 6, wherein:
said at least one band is located on an external surface of said article of clothing.

12. The firm-contact apparel prosthesis for tremor suppression according to claim 11, wherein:
said article of clothing is a glove; and
said at least one band is located in said palm area of said glove.

13. The firm-contact apparel prosthesis for tremor suppression according to claim 6, wherein:
said at least one band is located on an internal surface of said article of clothing.

14. The firm-contact apparel prosthesis for tremor suppression according to claim 13, wherein:
said article of clothing is a glove; and
said at least one band is located in said palm area of said glove.

15. The firm-contact apparel prosthesis for tremor suppression according to claim 7, wherein:
said external layer of said clothing has a first end and a second end;
said at least one band is located between said first end and said second end of said external layer.

16. A method of tremor suppression comprising the steps of:
providing an article of clothing;
providing at least one band of elastomeric material attached to said article of clothing;
locating said article of clothing with said band about the circumference of a body part; and
applying pressure around the entire circumference of the body part with said band.

17. The method of tremor suppression further comprising the step of:
applying said pressure for a desired number of time intervals.

18. The method of tremor suppression further comprising:
said article of clothing is a glove; and
said at least one band is located in said palm region of said glove.

19. The method of tremor suppression wherein:
said article of clothing is a sleeve.

20. A method of tremor suppression comprising the steps of:
providing an article of clothing;
providing a plurality of bands of elastomeric material attached to said article of clothing;
locating said article of clothing with said plurality of bands about the circumference of a body part; and
applying pressure around the entire circumference of the body part with said plurality of bands.

21. The method of tremor suppression further comprising the step of:
applying said pressure for a desired number of time intervals.
